(19) United States
(12) Patent Application Publication
(10) Pub. No.: US 2010/0036123 A1
(43) Pub. Date: Feb. 11, 2010

(54) PROCESS FOR THE PREPARATION OF 2,4-DICHLORO-7H-PYRROLO[2,3H]QUINAZOLINE

(75) Inventors: Kenneth Alfred Martin Kremer, Lawrenceville, NJ (US); Uresh Shantilal Shah, Cranbury, NJ (US); Aranapakam Mudumbai Venkatesan, Rego Park, NY (US); Zecheng Chen, New City, NY (US); Osvaldo dos Santos, Astoria, NY (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/537,307

(22) Filed: Aug. 7, 2009

Related U.S. Application Data
(60) Provisional application No. 61/086,890, filed on Aug. 7, 2008.

(51) Int. Cl. C07D 487/04

(52) U.S. Cl. 544/250

(57) ABSTRACT

The invention relates to a method of preparing 2,4-dichloro-7H-pyrrolo[2,3-h]quinazoline (II):

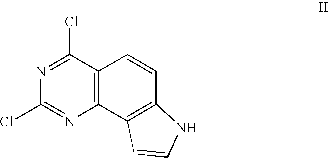

from 1H-pyrrolo[2,3-h]quinazoline-2,4-dione.

### FIELD OF THE INVENTION

The invention relates to an improved process for the preparation of 2,4-dichloro-7H-pyrrolo[2,3 h]quinazoline as an intermediate in the manufacture of biologically active mTOR and PI3K inhibitors.

### BACKGROUND OF THE INVENTION

Certain 7H-pyrrolo[2,3-h]quinazoline compounds, such as those disclosed in U.S. application Ser. No. 12/397,590 filed Mar. 4, 2009 (the disclosure of which is hereby incorporated herein by reference in its entirety), inhibit the enzymes mTOR and PI3K. They are useful in preventing and treating mTOR-related disorders and PI3K-related disorders including, disorders with which abnormal cell growth is associated. Examples of such abnormal cell growth disorders are restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the 7H-pyrrolo[2,3 h]quinazoline compounds possess excellent cancer cell growth inhibiting effects and are effective in treating cancers, preferably all types of solid cancers and malignant lymphomas, and especially, leukemia, skin cancer, bladder cancer, breast cancer, uterine cancer, ovarian cancer, prostate cancer, lung cancer, colon cancer, pancreas cancer, renal cancer, gastric cancer, brain tumor, etc.

The earlier filed provisional patent application describes the preparation of 2-aryl-7H-pyrrolo[2,3-h]quinazoline compounds from nitro indole compounds by reduction to the amine, cyclization and subsequent hydrolysis to the 1H-pyrrolo[2,3-h]quinazoline-2,4(3H,7H)-dione, conversion of carbonyl groups to the 2,4-dihalo-7H-pyrrolo[2,3 h]quinazoline, and Suzuki coupling at the 2-position to the final compound (see Schemes 1-6, U.S. Ser. No. 12/397,590 filed Mar. 4, 2009, Wyeth Case AM102868). The synthesis described herein eliminates the need to isolate the intermediates, uses less hydrogenation catalyst, eliminates the use of toxic halogenated solvents, shortens the reaction times, and eliminates a distillation step.

7H-Pyrrolo[2,3 h]quinazoline compounds are known to be intermediates compounds in the synthesis of 5-hydroxytryptamine-6 ligands. For example, in the following US patent application US 2007/0099911 A1 the synthesis of 4H-pyrrolo[2,3-h]quinazolin-4-one compounds is taught. The compounds described in this patent application lack the two oxygen atoms at positions 2 and 4 of the 1H-pyrrolo[2,3-h]quinazoline-2,4(3H,7H)-dione ring or the two halogen atoms at positions 2 and 4 of the 7H-pyrrolo[2,3 h]quinazoline ring of the present invention. 7H-Pyrrolo[2,3-h]quinazolin-2-amine and 7H-pyrrolo[2,3-h]quinazolin-2-one compounds are known to be intermediates in the synthesis of size-expanded DNA molecules. For example, Krueger et al, (Accounts of Chemical Research, 40(2), 141-150, 2007) teach the synthesis of these compounds. The compounds described in this reference have an amino group attached at position 2 or position 4 of the 7H-pyrrolo[2,3 h]quinazoline ring. 2-Amino-7H-pyrrolo[2,3 h]quinazoline compounds are known to be intermediates compounds in the synthesis of size-expanded base pairs. For example, Lu et al (Angewandte Chemie, International Edition, 43(43), 5834-5836, 2004) teach the synthesis of a number of such compounds containing a deoxyribose radical on position 7. The compounds described in this reference have an amino group attached at position 2 of the 7H-pyrrolo[2,3 h]quinazoline ring. Thus, the compounds and their uses described in these references are distinct from those compounds made by the processes of the present invention.

### SUMMARY OF THE INVENTION

In one aspect, the invention provides a method of preparing 1H-pyrrolo[2,3-h]quinazoline-2,4-dione (I):

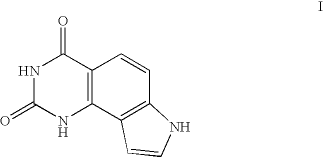

from 4-nitro-1H-indole.

In one aspect, the invention provides a method of preparing 2,4-dichloro-7H-pyrrolo[2,3-h]quinazoline (II):

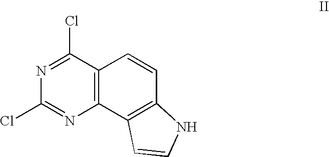

from 1H-pyrrolo[2,3-h]quinazoline-2,4-dione (I).

In one aspect, the invention provides a method of preparing 2,4-dichloro-7-(phenylsulfonyl)-7H-pyrrolo[2,3-h]quinazoline (IV):

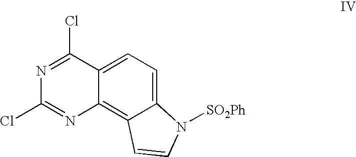

from 7-(phenylsulfonyl)-1H-pyrrolo[2,3-h]quinazoline-2,4(3H,7H)-dione.

### DETAILED DESCRIPTION OF THE INVENTION

One aspect of the invention provides a method of preparing 1H-pyrrolo[2,3-h]quinazoline-2,4-dione (I):

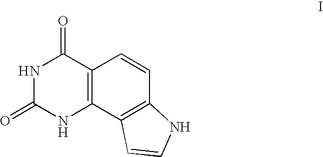

from 4-nitro-1H-indole comprising the steps of reacting 4-nitro-1H-indole with an amine-protecting agent, reducing the nitro group of the product of said reaction to an amine, and reacting said amine with an C₁-C₆alkyl, C₂-C₆alkenyl, C₂-C₆alkynyl, C₆-C₁₄aryl, C₃-C₈cycloalkyl, or C₁-C₉heteroaryl isothiocyanatoformate to yield a 4-thioureido-1H-indole in an aromatic hydrocarbon solvent.

In one aspect, said aromatic hydrocarbon solvent is toluene.

In one aspect, the steps of reacting 4-nitro-1H-indole with a amine-protecting agent, reducing the nitro group of product of that reaction to an amine, and reacting said amine with an C₁-C₆alkyl, C₂-C₆alkenyl, C₂-C₆alkynyl, C₆-C₁₄aryl, C₃-C₈cycloalkyl, or C₁-C₉heteroaryl isothiocyanatoformate to yield a 4-thioureido-1H-indole are done without isolation of intermediates.

In one aspect, said amine-protecting agent transfers a t-butoxycarbonyl group to the indole nitrogen atom.

In one aspect, said amine-protecting agent is BOC₂O.

In one aspect, said reducing is done catalytically.

In one aspect, said catalytic reducing is done with hydrogen.

In one aspect, said catalytic reducing is done with hydrogen and Pd—C.

In one aspect, said Pd—C is 5% by weight and is 50% water wet.

In one aspect, said isothiocyanatoformate is an C₁-C₆alkyl isothiocyanatoformate.

In one aspect, said C₁-C₆alkyl isothiocyanatoformate is ethyl isothiocyanatoformate.

In one aspect, the invention further comprises reacting the 4-thioureido-1H-indole with an alkylating agent R²—X, wherein R² is C₁-C₆alkyl and X is a leaving group to give a carbonylamino(C₁-C₆alkylthio)methyleneamino)-1H-indole.

In one aspect, said R²—X is ethyl iodide.

In one aspect, the invention further comprises induction of cyclization of the carbonylamino(C₁-C₆alkylthio)methyleneamino)-1H-indole to give a 2-thio-3H-pyrrolo[2,3-h]quinazolin-4(7H)-one.

In one aspect, said induction of cyclization of the carbonylamino(C₁-C₆alkylthio)methyleneamino)-1H-indole intermediate group is done thermally.

In one aspect, the thermal cyclization is done in a eutectic mixture of biphenyl (C₁₂H₁₀) and diphenyl oxide (C₁₂H₁₀O).

In one aspect, the invention further comprises hydrolysis of the 2-thio-3H-pyrrolo[2,3-h]quinazolin-4(7H)-one to give the final product.

In one aspect, said hydrolysis is done in an acidic medium.

In one aspect, the invention provides a method of preparing 2,4-dichloro-7H-pyrrolo[2,3-h]quinazoline (II):

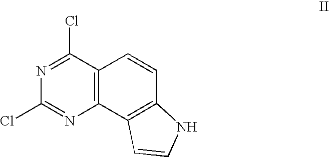

from 1H-pyrrolo[2,3-h]quinazoline-2,4-dione comprising the step of reacting 1H-pyrrolo[2,3-h]quinazoline-2,4-dione with a chlorinating agent, wherein said 1H-pyrrolo[2,3-h]quinazoline-2,4-dione has a purity >97 area % by HPLC.

In one aspect, the invention provides a method of preparing 2,4-dichloro-7-(phenylsulfonyl)-7H-pyrrolo[2,3-h]quinazoline (IV):

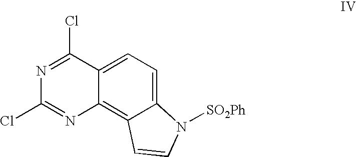

from 7-(phenylsulfonyl)-1H-pyrrolo[2,3-h]quinazoline-2,4(3H,7H)-dione (III):

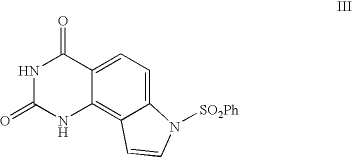

comprising the step of reacting III with a chlorinating agent.

In one aspect, the invention further comprises the step of hydrolysis of a 2-(C₁-C₆alkylthio)-7-(phenylsulfonyl)-3H-pyrrolo[2,3-h]quinazolin-4(7H)-one to give III.

In one aspect, the invention further comprises the steps of reacting 4-nitro-1H-indole with benzenesulfonyl chloride, reducing the nitro group of the product of said reaction to an amine, reacting said amine with an C₁-C₆alkyl, C₂-C₆alkenyl, C₂-C₆alkynyl, C₆-C₁₄aryl, C₃-C₈cycloalkyl, or C₁-C₉heteroaryl isothiocyanatoformate to yield a 4-thioureido-1H-indole, reacting the 4-thioureido-1H-indole with an alkylating agent R²—X, wherein R² is C₁-C₆alkyl and X is a leaving group to give a carbonylamino(C₁-C₆alkylthio)methyleneamino)-1H-indole, and induction of cyclization of the carbonylamino(C₁-C₆alkylthio)methyleneamino)-1H-indole to give a 2-(C₁-C₆alkylthio)-7-(phenylsulfonyl)-3H-pyrrolo[2,3-h]quinazolin-4(7H)-one.

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-pu rine-2,6-dione), triethiodide, tromethamine (2-amino-2-(hydroxymethyl)-1,3-propanediol), valerate, and zinc salts.

An “effective amount” when used in connection a compound of the present invention of this invention is an amount effective for inhibiting mTOR or PI3K in a subject.

### DEFINITIONS

The following definitions are used in connection with the compounds of the present invention unless the context indicates otherwise. In general, the number of carbon atoms present in a given group is designated “Cₓ-C_{y}” where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are arrived at by naming from left to right the terminal portion of the functionality followed by the adjacent functionality toward the point of attachment. For example, the substituent “arylalkyloxycabonyl” refers to the group (C₆-C₁₄aryl)-(C₁-C₆alkyl)-O—C(O)—. It is understood that the above definitions are not intended to include impermissible substitution patterns (e.g., methyl substituted with 5 fluoro groups, two hydroxyl groups on a single carbon atom, a hydroxyl group on a non-aromatic double bond). Such impermissible substitution patterns are well known to the skilled artisan. In each of the below groups, when a subgroup is designated with a multiple occurrence, each occurrence is selected independently. For example, in di(C₁-C₆alkyl)amino- e.g. (C₁-C₆alky)₂N—, the C₁-C₆alky groups can be the same or different.

“Alkenyl-” refer to a straight or branched chain unsaturated hydrocarbon containing at least one double bond. Where E- and/or Z-isomers are possible, the term “alkenyl” is intended to include all such isomers. Examples of a C₂-C₆alkenyl-group include, but are not limited to, ethylene, propylene, 1-butylene, 2-butylene, isobutylene, sec-butylene, 1-pentene, 2-pentene, isopentene, penta-1,4-dien-1-yl, 1-hexene, 2-hexene, 3-hexene, and isohexene. An alkenyl-group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, and C₃-C₈cycloalkyl-.

“Alkyl-” refers to a hydrocarbon chain that may be a straight chain or branched chain, containing the indicated number of carbon atoms, for example, a C₁-C₁₀alkyl-group may have from 1 to 10 (inclusive) carbon atoms in it. In the absence of any numerical designation, “alkyl-” is a chain (straight or branched) having 1 to 6 (inclusive) carbon atoms in it. Examples of C₁-C₆alkyl-groups include, but are not limited to, methyl, ethyl, propyl, butyl, pentyl, hexyl, isopropyl, isobutyl, sec-butyl, tert-butyl, isopentyl, neopentyl, and isohexyl. An alkyl-group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carboxyamido-, HC(O)NH—, H₂NC(O)—, (C₁-C₆alkyl)NHC(O)—, di(C₁-C₆alkyl)NC(O)—, NC—, hydroxyl-, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxyl-, C₁-C₆carboxyamidoalkyl-, or O₂N—.

“Alkylthio-” refers to the group R—S— where R is an alkyl group, as defined above, attached to the parent structure through a sulfur atom. Examples of C₁-C₆alkylthio-include methylthio, ethylthio, n-propylthio, i-propylthio, n-butylthio, i-butylthio, s-butylthio, t-butylthio, n-pentylthio and n-hexylthio.

“Alkynyl-” refers to a straight or branched chain unsaturated hydrocarbon containing at least one triple bond. Examples of a C₂-C₆alkynyl-group include, but are not limited to, acetylene, propyne, 1-butyne, 2-butyne, isobutyne, sec-butyne, 1-pentyne, 2-pentyne, isopentyne, penta-1,4-diyn-1-yl, 1-hexyne, 2-hexyne, 3-hexyne, and isohexyne. An alkynyl group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, and C₃-C₈cycloalkyl-.

“Amine-protecting agent” refers to a reactive reagent capable of transferring an amine-protecting group to a nitrogen atom in the target molecule. The amine-protecting group is capable of surviving subsequent chemical reactions applied to the target molecule i.e. hydrogenation, reaction with acylating agents, alkylation etc. The amine-protecting group can later be removed. Examples of an amine-protecting agent include, but are not limited to, C₁-C₆ aliphatic acid chlorides or an hydrides, C₆-C₁₄arylcarboxylic acid chlorides or anhydrides, t-butyl chloroformate, di-tert-butyl dicarbonate, butoxycarbonyloxyimino-2-phenylacetonitrile, t-butoxycarbonyl azide, t-butyl fluoroformate, fluorenylmethoxycarbonyl chloride, fluorenylmethoxycarbonyl azide, fluorenylmethoxycarbonyl benzotriazol-1-yl, fluorenylmethoxycarbonyl succinimidyl, fluorenylmethoxycarbonyl pentafluorophenoxide, trichloroacetyl chloride, methyl-, ethyl-, trichloromethyl-chloroformate, and other amine protecting agents known in the art. Examples of such known amine-protecting agents are found in pages 385-397 of T. W. Green, P. G. M. Wuts, “Protective Groups in Organic Synthesis, Second Edition”, Wiley-Interscience, New York, 1991. Amine protecting groups include, but are not limited to, fluorenylmethoxycarbonyl (FMOC), tert-butoxycarbonyl (t-BOC), those of the acyl type (e.g., formyl, benzoyl, trifluoroacetyl, p-tosyl, aryl- and alkylphosphoryl, phenyl- and benzylsulfonyl, o-nitrophenylsulfenyl, o-nitrophenoxyacetyl), and of the urethane type (e.g. tosyloxyalkyloxy-, cyclopentyloxy-, cyclohexyloxy-, t-butyloxy, 1,1-dimethylpropyloxy, 2-(p-biphenyl)-2-propyloxy- and benzylthiocarbonyl).

“Aromatic hydrocarbon solvent” refers to a liquid unsaturated cyclic hydrocarbon containing one or more rings which has at least one 6-carbon ring containing three double bonds. It is capable of dissolving a solute to form a uniformly dispersed solution. Examples of an aromatic hydrocarbon solvent include, but are not limited to, benzene toluene, ethylbenzene, m-xylene, o-xylene, p-xylene, indane, naphthalene, trimethylbenzene, chlorobenzene, fluorobenzene, trifluorotoluene, anisole or mixtures thereof.

Aryl-refers to an aromatic hydrocarbon group. Examples of an C₆-C₁₄aryl-group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl. An aryl group can be monocyclic or polycyclic as long as at least one ring is aromatic and the point of attachment is at an aromatic carbon atom. An aryl group can be unsubstituted or substituted with one or more of the following groups: C₁-C₆alkyl-, halogen, haloalkyl-, hydroxyl, hydroxyl (C₁-C₆alkyl)-, H₂N—, aminoalkyl-, di(C₁-C₆alkyl)amino-, HO₂C—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Chlorinating agent” refers to various inorganic and organic reagents having the functionality of an acid chloride. Examples of a chlorinating agent include, but are not limited to, antimony trichloride, n-chlorosuccinimide, ferric chloride, nitryl chloride, oxalyl chloride, phosphorus oxychloride, phosphorus pentachloride, phosphorus trichloride, sulfur dichloride, sulfuryl chloride, phosgene; oxalyl chloride, chloromethylenedimethylammonium chloride, and thionyl chloride.

“Cycloalkyl-” refers to a monocyclic saturated hydrocarbon ring. Representative examples of a C₃-C₈cycloalkyl-include, but are not limited to, cyclopropyl, cyclobutyl, cyclopentyl, cyclohexyl, cycloheptyl, and cyclooctyl. A cycloalkyl-can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, or C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—. Additionally, each of any two hydrogen atoms on the same carbon atom of the carbocyclic ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle-containing two oxygen atoms.

“Halo” or “halogen” refers to fluorine, chlorine, bromine, or iodine.

“Heteroaryl-” refers to 5-10-membered mono and bicyclic aromatic groups containing at least one heteroatom selected from oxygen, sulfur, and nitrogen, wherein any S can optionally be oxidized, and any N can optionally be quaternized with an C₁-C₆alkyl group. Examples of monocyclic C₁-C₉heteroaryl-radicals include, but are not limited to, oxazinyl, thiazinyl, diazinyl, triazinyl, thiadiazolyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of bicyclic C₁-C₉heteroaryl-radicals include but are not limited to, benzimidazolyl, indolyl, isoquinolinyl, benzofuranyl, benzothiophenyl, indazolyl, quinolinyl, quinazolinyl, purinyl, benzisoxazolyl, benzoxazolyl, benzthiazolyl, benzodiazolyl, benzotriazolyl, isoindolyl, and indazolyl. The contemplated heteroaryl-rings or ring systems have a minimum of 5 members. Therefore, for example, C₁heteroaryl-radicals would include but are not limited to tetrazolyl, C₂heteroaryl-radicals include but are not limited to triazolyl, thiadiazolyl, and tetrazinyl, C₉heteroaryl-radicals include but are not limited to quinolinyl and isoquinolinyl. A heteroaryl-group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆aminoalkyl-, di(C₁-C₆alkyl)amino-, —COOH, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Isothiocyanatoformate” refers to a compound of the formula R—O—C(O)—N═C═S, where R is a C₁-C₆alkyl, C₂-C₆alkenyl, C₂-C₆alkynyl, C₆-C₁₄aryl, C₃-C₈cycloalkyl, or C₁-C₉heteroaryl radical.

The term “optionally substituted”, unless otherwise specified, as used herein means that at least one hydrogen atom of the optionally substituted group has been substituted with halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carboxyamido-, HC(O)NH—, H₂NC(O)—, (C₁-C₆alkyl)NHC(O)—, di(C₁-C₆alkyl)NC(O)—, NC—, hydroxyl-, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, or C₃-C₈cycloalkyl-.

Procedures used to synthesize the compounds of the present invention are described in Schemes 1-4 and are illustrated in the examples. Reasonable variations of the described procedures are intended to be within the scope of the present invention:

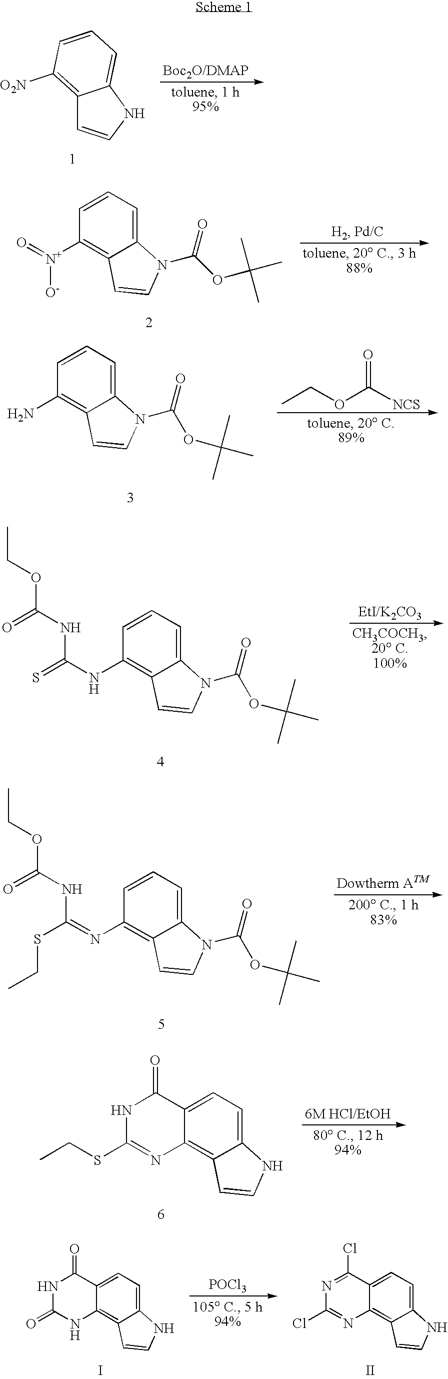

As shown in Scheme 1, the solvent was changed to aromatic hydrocarbon from the methylene chloride used in the process of U.S. Ser. No. 12/397,590. An alternate solvent is THF. The reaction concentration was increased from about 8% to 13%. The BOC₂O was added in portions vs. one charge. In this procedure the product 2 was isolated by filtration rather than concentration to dryness in the procedure of U.S. Ser. No. 12/397,590. The reaction was complete at end of BOC₂O addition. The solvent for the hydrogenation was changed to toluene from the ethanol used in the process of U.S. Ser. No. 12/397,590, same as new step 1 and 3. The 10% wt % dry Pd—C catalyst used in the process of U.S. Ser. No. 12/397,590 was replaced with 5% wt % wet Pd—C catalyst and usage reduced by 90%. Much less catalyst is required on scale up and this minimizes over hydrogenation to indolene 12. Alternately 5 wt % Pd Degussa Type E 1002 U/W™ catalyst gave acceptable results. The results are comparable to the 10 wt % Pd—C in EtOH used in U.S. Ser. No. 12/397,590. Solvent changed to toluene from the methylene chloride used in the process of U.S. Ser. No. 12/397,590, same as new step 1 and 2. The product of step 3 unexpectedly precipitated from toluene reaction mixture. The product 4 was isolated by simple filtration from the toluene reaction mixture vs. the procedure of concentration to dryness of the methylene chloride solvent used in U.S. Ser. No. 12/397,590. This eliminated the halogenated solvent for step 4 in work-up and replaced it with ethyl acetate.

The solvent in step 5 was changed to Dowtherm ATM from the diphenyl ether used in the process of U.S. Ser. No. 12/397,590. The new procedure simultaneously cyclizes and hydrolyzes the BOC group to form new reaction intermediate 6 rather than tert-butyl 2-(ethylthio)-4-oxo-3,4-dihydro-7H-pyrrolo[2,3-h]quinazoline-7-carboxylate obtained previously. The reaction concentration increased to 11% from 5%. The intermediate 6 unexpectedly precipitates as a massive well stirring slurry during reaction and is isolated by filtration. The reaction time was cut from 5 hour to 1 hour and the volatile hexane wash solvent was replaced by heptane. The yield increased from 46% to 83%.

In step 6, the reaction concentration was increased to 5% from the 2.5% obtained in U.S. Ser. No. 12/397,590. The distillation step was eliminated in new procedure: Product I unexpectedly precipitated during reaction and is isolated by filtration vs. distillation required in the original procedure. The yield increased from 58% to 94% in the final step since the starting material was of high purity due to alternate improved isolation procedure.

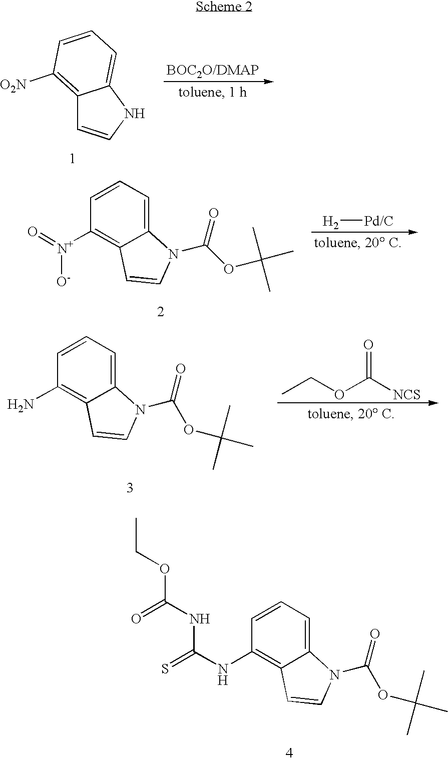

The integrated process developed combining Steps 1 to 3 using a single solvent, toluene, is shown in Scheme 2. This eliminated the two product isolations in Steps 1 and 2. The non-optimized three step overall real yield is ca. 66% vs. 74% for the individually run steps.

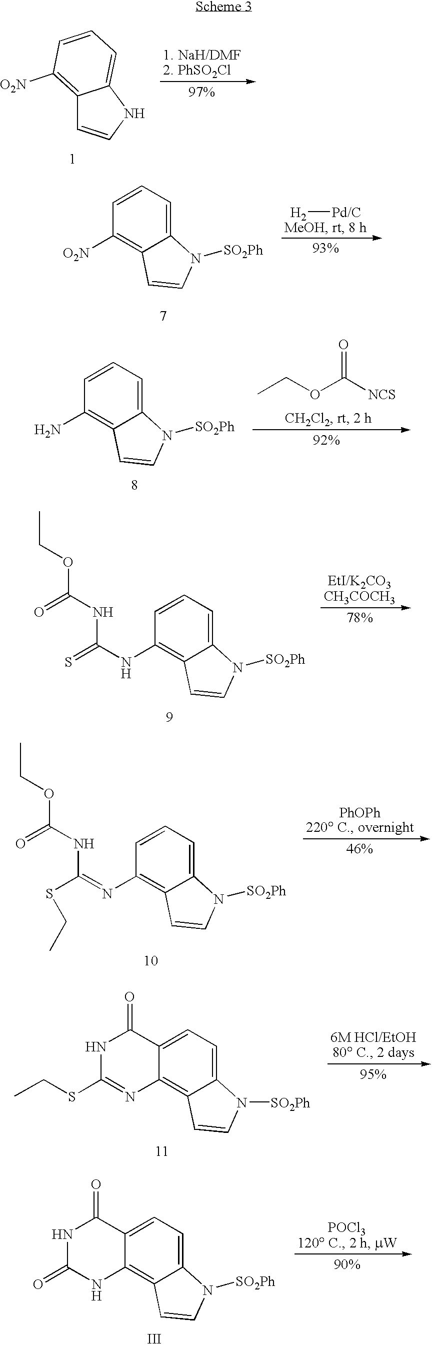

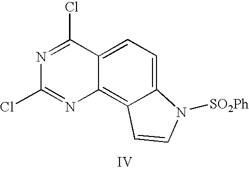

The synthesis of compounds III and IV from 4-nitro-1H-indole (1) is shown in Scheme 3.

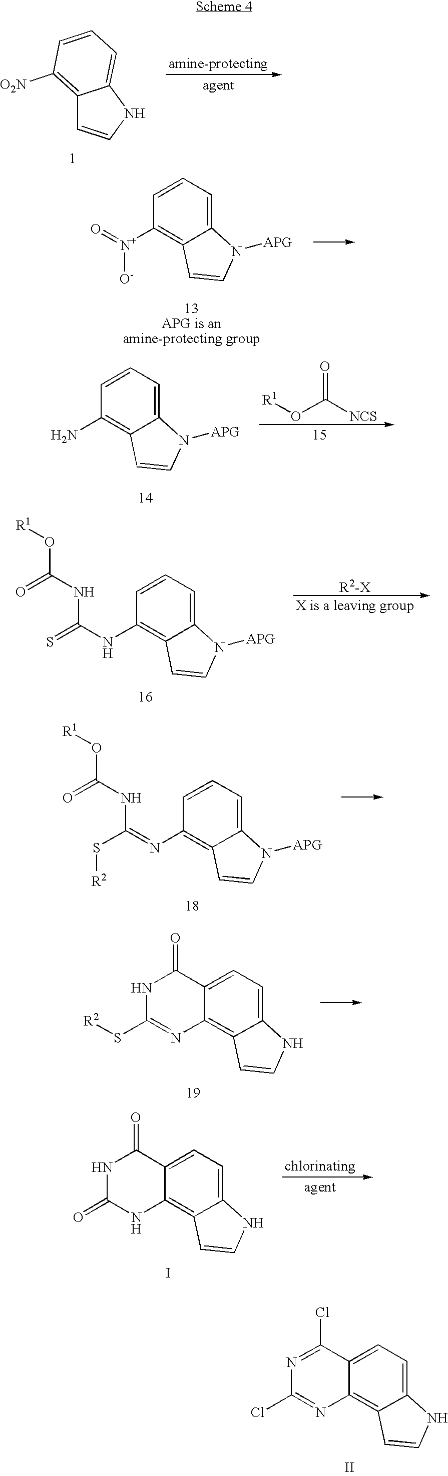

Synthesis of I and II is shown in Scheme 4. R¹ is C₁-C₆alkyl, C₂-C₆alkenyl, C₂-C₆alkynyl, C₆-C₁₄aryl, C₃-C₈cycloalkyl, or C₁-C₉heteroaryl and R² is C₁-C₆alkyl.

One of skill in the art will recognize that Schemes 1-4 can be adapted to produce the other compounds of Formula I and pharmaceutically acceptable salts of compounds of Formula I according to the present invention.

### EXAMPLES

The following abbreviations are used herein and have the indicated definitions: BOC is the tertiary-butyloxycarbonyl group, BOC₂O is di-tert-butyl dicarbonate, CAN is acetonitrile, and Celite™ is flux-calcined diatomaceous earth. Celite™ is a registered trademark of World Minerals Inc. Chromolith™ consists of a single piece of high-purity polymeric silica gel with a bimodal pore structure of 2 μm macropores and 13 nm mesopores. Chromolith™ is a registered trademark of Merck KGaA, Darmstadt, Germany. Degussa Type E101 NO/W™ (50% water wet) is a palladium on activated carbon catalyst with a particle size of 20 μm, containing 5% precious metal, and 5) % water. E101 NO/W™ is a registered trademark of Evonik Industries AG. DMAP is 4-(dimethylamino)pyridine. Dowtherm A™ is a eutectic mixture of biphenyl (C₁₂H₁₀) and diphenyl oxide (C₁₂H₁₀O). Dowtherm A™ is a registered trademark of Dow Corning Corporation. DMF is N,N-dimethylformamide, EtOAc is ethyl acetate, and EtOH is ethanol. HPLC is high-pressure liquid chromatography, MeOH is methanol, MS is mass spectrometry, mTOR is Mammalian Target of Rapamycin (a protein), and PI3K is phosphoinositide 3-kinase (an enzyme). TFA is trifluoroacetic acid.

### Synthetic Methods

The following methods outline the Examples of the present invention.

### Integrated Compound 1 to 4 Procedure:

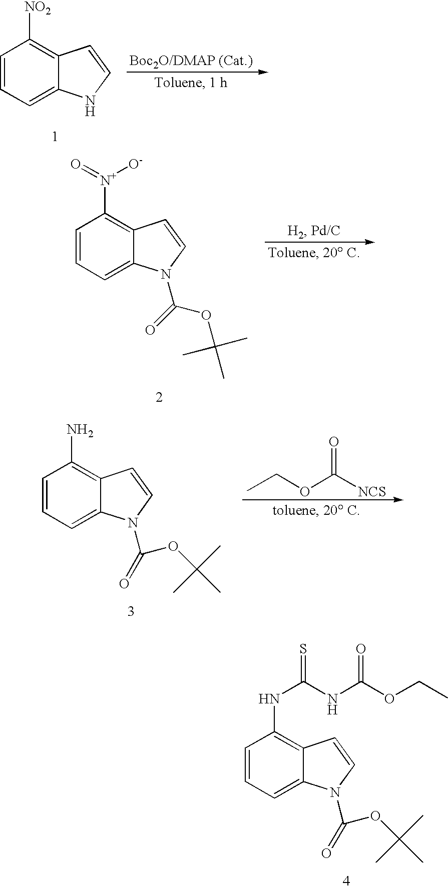

### Step 1: Preparation of 4-nitro-indole-1-carboxylic acid tert-butyl ester (2)

To a 1 L four-necked round bottom flask equipped with mechanical stirrer, nitrogen inlet, thermocouple and condenser was charged 18.1 g (0.111 mol) of 4-nitroindole, 140 mL toluene and 0.27 g of DMAP. At room temperature, solid BOC₂O (26.6 g 0.122 mol, 1.10 eq) was added in portions over about 45 minutes to the stirring mixture while maintaining moderate gas evolution. After stirring the resulting solution for 10 minutes, HPLC analysis showed the reaction to be complete with no starting material. The batch was quenched with 50 mL water and the phases were split in a separatory funnel. The organic layer was heated to remove water by azeotrope formation and then concentrated to give a 236 gram toluene solution of the product. This solution was used directly in step 2.

### Step 2: Preparation of 4-amino-indole-1-carboxylic acid tert-butyl ester (3)

To a 500 mL Paar shaker hydrogenation flask was charged 118.0 g of toluene solution from Step 1 containing (14.6 g, 0.0555 mol) of 4-nitro-indole-1-carboxylic acid tert-butyl ester (2) and 0.36 g of 5 wt % Pd—C Degussa Type E101 NO/W™ (50% water wet). The yellow-green mixture was hydrogenated at about 50 psi at room temperature for about 7 hours until no further hydrogen uptake and HPLC analysis showed the reaction to be complete. Note: Batch should be analyzed quickly when hydrogen uptake is complete since indolene by-product forms due to over hydrogenation. (Extended reaction time results in yield loss via over hydrogenation). The mixture was filtered through Celite™, washed with toluene, dried by azeotrope distillation, and concentrated to a batch weight of 120 grams of toluene solution of product. This solution was used directly in Step 3.

### Step 3: Preparation of tert-butyl 4-(3-ethoxycarbonyl)thioureido)-1H-indole-1-carboxylate (4)

To a 1 L four-necked flask equipped with a mechanical stirrer, thermocouple and N₂ inlet tube was added 120 gram of toluene solution of tert-butyl 4-amino-1H-indole-1-carboxylate (12.9 g, 0.0555 mole). To this clear, stirred solution, ethyl isothiocyanatoformate (7.3 g, 0.0555 mole, 1.0 eq) was added over one hour period keeping internal temperature between 20-28° C. The product precipitated during the addition. HPLC analysis showed the reaction was complete at the end of the addition. The product was isolated by filtration and washed with 1:1 toluene:heptane (150 ml) to give 15.8 g pale yellow product. The purity by HPLC was about 89% and the overall real yield from Step 1 to 3 corrected for the purity was 66%. Crude overall three-step yield is 79%.

### Stepwise Procedures for the Improved Process

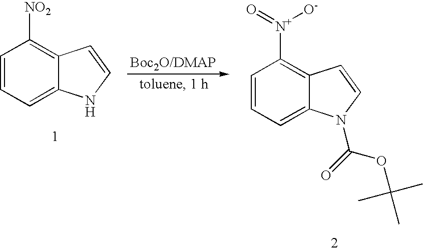

### Step 1: Preparation of 4-Nitro-indole-1-carboxylic acid tert-butyl ester (2)

To a 5 L four-necked round bottom flask equipped with mechanical stirrer, nitrogen inlet, thermocouple, and condenser was charged 182.3 g (1.124 mol) of 4-nitroindole, 1400 mL toluene, and 2.7 g of DMAP. At room temperature, solid BOC₂O (270.0 g 1.237 mol, 1.10 eq) was added in portions over about 45 minutes to the stirring mixture while maintaining moderate gas evolution. After stirring the resulting solution for 1 hour, HPLC analysis showed the reaction to be complete with no starting material. The batch was quenched with 500 mL water, transferred to a separatory funnel and the phases were split. The organic layer was washed with 500 mL brine, concentrated to a slurry, diluted with 1000 ml heptane, filtered, and washed with fresh heptane. The filter cake was dried under vacuum at RT to constant weight to afford 206.0 gram of pale yellow solid. A second crop of 20.0 gram of product was isolated as post-precipitate from the mother liquors. The overall yield was 226.0 gram, 95.8% yield of pale yellow solid (100 area % HPLC).

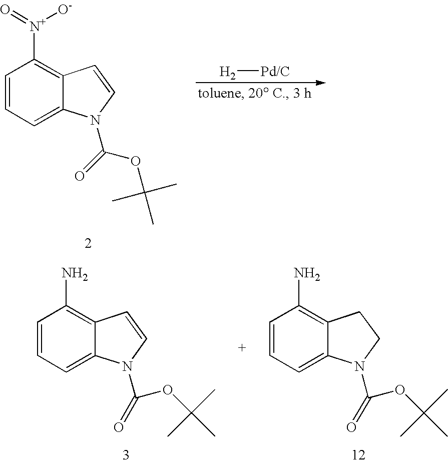

### Step 2: Preparation of 4-amino-indole-1-carboxylic acid tert-butyl ester (3)

To a 2.5 liter Paar shaker hydrogenation flask was charged 40.0 g (0.152 mol) of, 500 mL of toluene and 1.0 g of 5 wt % Pd—C Degussa Type E101 NO/W™ (50% water wet). The yellow-green mixture was hydrogenated at about 50 psi at room temperature for about 3 hours until no further hydrogen uptake and HPLC analysis showed the reaction to be complete. Note: batch should be analyzed quickly when hydrogen uptake is complete since about 5-15% of indolene by-product forms due to over hydrogenation. (Extended reaction time results in yield loss via over hydrogenation). The mixture was filtered through Celite™, washed with toluene, and concentrated.

Multiple hydrogenation batches were combined and concentrated to 140 gram crude orange oil. The material was purified by chromatography with 25% EtOAc: 75% hexane and eluted to afford 121 gram of pure product as an orange oil in 100 area % by HPLC in an overall yield of 88%. The indolene by-product 12 must be removed in this step otherwise it carries through to later steps.

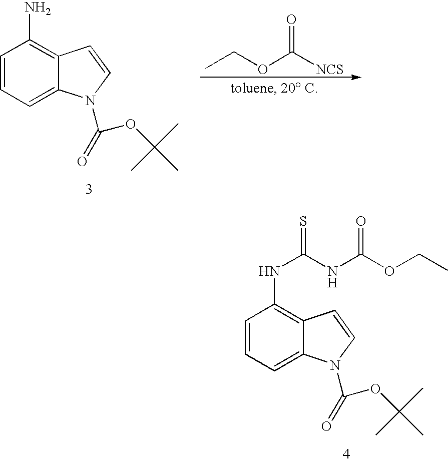

### Step 3: Preparation of tert-butyl 4-(3-ethoxycarbonyl)thioureido)-1H-indole-1-carboxylate (4)

To a 3 L four-necked flask equipped with a mechanical stirrer, thermocouple, and N₂ inlet tube was added tert-butyl 4-amino-1H-indole-1-carboxylate (121.0 g, 0.521 mole) and toluene (1100 mL). The reaction mixture was stirred at room temperature and a clear solution was formed. To this clear solution, ethyl isothiocyanatoformate (68.3 g, 0.52 mole, 1.0 eq) was added over one hour period keeping internal temperature between 20-28° C. The product precipitated during the addition. HPLC analysis showed the reaction was complete at the end of the addition. The product was filtered off and washed with 1:1 toluene:heptane (800 ml) to give 169.0 g light yellow product in 89.4% yield (100% pure by analytical HPLC)

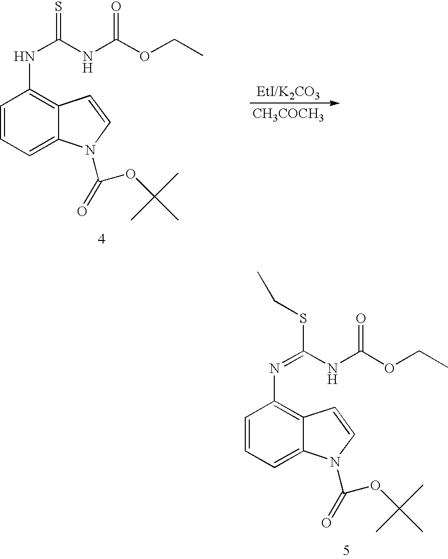

### Step 4: Preparation of (E)-tert-butyl 4-((ethoxycarbonylamino)(ethylthio)methyleneamino)-1H-indole-1-carboxylate (5)

To a 12 L four-necked flask equipped with a mechanical stirrer, N₂ inlet tube, and an addition funnel was added tert-butyl 4-(3-ethoxycarbonyl)thioureido)-1H-indole-1-carboxylate (4, 179 g, 0.493 mole) and acetone (7 L). To this clear solution, K₂CO₃ (136 g, 0.986 mole, 2.0 eq) was added. The reaction mixture was stirred at room temperature and ethyl iodide (84 g, 0.538 mole. 1.1 eq) was added by drops over 1 hours. The reaction mixture was stirred at room temperature for 20 hours. HPLC analysis showed <3% non-reacted 4 left. The reaction mixture was filtered to remove inorganic salts, concentrated to a slurry, diluted with ethyl acetate (4 L), washed with water (1 L), washed with brine (1 L), dried over anhydrous sodium sulfate, filtered, and concentrated to give 192 g product as a thick yellow oil in 100% yield (90 area % HPLC). It was used as-is in the next reaction.

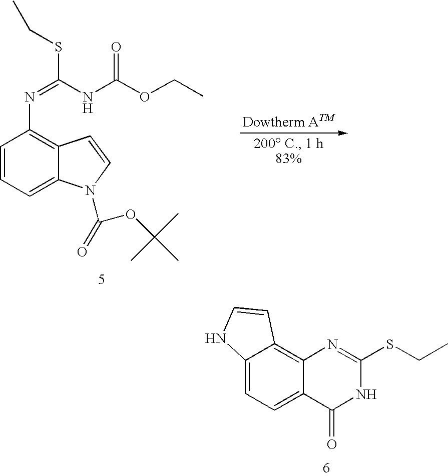

### Step 5: Preparation of 2-(ethylthio)-3H-pyrrolo[2,3-h]quinazolin-4(7H)-one (6)

To a 5 L four-necked flask equipped with mechanical stirrer, N₂ inlet tube, thermocouple was added 194.0 g (0.496 mol) of (E)-tert-butyl-4-[(ethoxycarbonylamino)(ethylthio)methyleneamino)]-1H-indole-1-carboxylate 5 and 1700 mL Dowtherm A™. The resulting slurry was heated to 200° C. and became a clear solution. The clear solution turned to a massive slurry as the product forms and the reaction mixture was held for 1 hour at 200° C. HPLC analysis showed complete conversion to product. The reaction mixture was cooled and diluted with heptane (3 L). The slurry was filtered, washed with heptane, and dried under suction to give 100 g product as a yellow solid in 83% yield. Analytical HPLC purity 97-98%

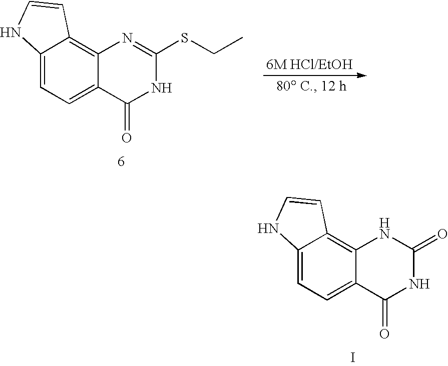

### Step 6: Preparation of 1H-pyrrolo[2,3-h]quinazoline-2,4(3H,7H)-dione (I)

To a 5 L four-necked flask equipped with mechanical stirrer, reflux condenser, and a thermocouple was added 93.7 gram (0.382 mol) of 2-(ethylthio)-3H-pyrrolo[2,3-h]quinazolin-4(7H)-one 6, ethanol (950 mL), and 6N HCl (950 mL). The reaction solution was heated to 80-86° C. for 12 hours. HPLC analysis showed 2-3% non-cleaved tert-butyl 2-(ethylthio)-4-oxo-3,4-dihydro-7H-pyrrolo[2,3-h]quinazoline-7-carboxylate carried over from the previous step. The product precipitated as a slurry during the reaction and was filtered hot and washed with water. The crude cake was dried under vacuum at 70° C. for 18 hour until constant weight to afford 72.3 g of beige solid in 94% yield (>97 area % HPLC).

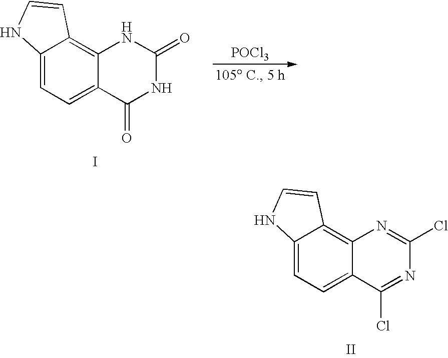

### Step 7: Preparation of 2,4-Dichloro-7H-pyrrolo[2,3-h]quinazoline (II)

To a 2 L four-necked flask equipped with a mechanical stirrer, N₂ inlet tube, reflux condenser, thermocouple, and a scrubber was added 1H-pyrrolo[2,3-h]quinazoline-2,4(3H,7H)-dione (1.30 g, 0.149 mole) and POCl₃ (600 mL). The reaction mixture was heated to 105-107° C. for 5 h and became a clear solution. HPLC analysis showed no I was left. The reaction mixture was cooled to room temperature and excess POCl₃ was removed by distillation under reduce pressure. The residue was quenched with ice/water to afford yellow precipitate that was filtered and triturated with hot ethanol to give 33 g product in 94% yield (>95 area % HPLC).

HPLC analysis conditions were Chromolith C18 4.6×100 mm column, gradient 10-100% ACN in water (0.1% TFA) in 3.5 min, then wash with 100% ACN for 1.5 min, the flow rate was 4 mL/min and the detection was at 230 nm.

### Preparation of 2,4-dichloro-7-(phenylsulfonyl)-7H-pyrrolo[2,3-h]quinazoline (IV)

### Step 1: Synthesis of 4-nitro-1-(phenylsulfonyl)-1H-indole (7)

To a solution of 4-nitroindole (1, 4.0 g, 24.6 mmol) in DMF (20 mL) was added sodium hydride (60%, 1.48 g, 37 mmol) in portions. After being stirred at room temperature for 15 minutes, benzenesulfonyl chloride was added by drops at 0° C., and the resulting reaction mixture was stirred at room temperature for additional 2 hours. The reaction was quenched by addition of aqueous ammonium chloride solution (5 mL), followed by addition of water. The resulting pale-yellow solid was collected by filtration, washed with water, and dried in vacuum to give the product (7.2 g, 97% yield), MS (ESI) m/z 302.9.

### Step 2: Synthesis of ethyl ({[1-(phenylsulfonyl)-1H-indol-4-yl]amino}carbonothioyl)carbamate (9)

To a solution of 4-nitro-1-(phenylsulfonyl)-1H-indole (7, 4.7 g, 15.6 mmol) in MeOH (50 mL) was added 10% Pd—C (470 mg) under N₂. The resulting mixture was shaken under hydrogen (H₂, 50 psi) at room temperature for 8 hours. The mixture was filtered through a pad of Celite™, and washed with EtOH. The filtrate was concentrated under reduced pressure to give the corresponding reduction product 8 as syrup (3.93 g, 93% yield).

The above 4-amino compound 8 was dissolved in CH₂Cl₂ (100 mL), and ethyl isothiocyanatoformate (1.63 mL, 14.5 mmol) was added. The resulting mixture was stirred at room temperature overnight, and the solvent was removed in vacuum to give the title product 9 as yellow solid (5.39 g, 92% yield), MS (ESI) m/z 404.2.

### Step 3: Synthesis of 2-(ethylthio)-7-(phenylsulfonyl)-7H-pyrrolo[2,3-h]quinazolin-4-ol (11)

To a solution of ethyl ({[1-(phenylsulfonyl)-1H-indol-4-yl]amino}carbonothioyl)carbamate (9, 5.39 g, 13.4 mmol) in acetone (150 mL) was added K₂CO₃ (3.69 g, 26.7 mmol), followed by addition of iodoethane (1.1 mL, 13.4 mmol) at room temperature. The resulting mixture was vigorously stirred at room temperature overnight. The mixture was filtered and washed with acetone. The filtrate was concentrated under reduced pressure, and the residue was treated with CH₂Cl₂ and water. The mixture was extracted with CH₂Cl₂, and the extracts were washed with water, and dried over MgSO₄. The solvent was removed under reduced pressure to provide the corresponding ethylthio compound 10 as colorless oil (4.5 g, 78% yield), MS (ESI) m/z 432.3.

To the above product was added phenyl ether (70 mL), and the resulting mixture was heated at 220° C. overnight. The reaction mixture was cooled down to room temperature, and diluted with hexanes. The resulting solid was collected by filtration to give the title compound 11 as off-white solid (1.83 g, 46% yield), MS (ESI) m/z 386.3.

### Step 4: Synthesis of 7-(phenylsulfonyl)-1H-pyrrolo[2,3-h]quinazoline-2,4(3H,7H)-dione (III)

To a solution of 2-(ethylthio)-7-(phenylsulfonyl)-7H-pyrrolo[2,3-h]quinazolin-4-ol (11, 1.83 g, 4.75 mmol) in EtOH (30 mL) was added 6N HCl aqueous solution (30 mL) at room temperature. The resulting mixture was heated at 80° C. for 2 days. The reaction mixture was cooled down to room temperature, and concentrated under reduced pressure to its half volume. The resulting solid was collected by filtration to give title product III as off-white solid (1.546 g, 95% yield), MS (ESI) m/z 340.1 [M-1].

### Step 5: Synthesis of 2,4-dichloro-7-(phenylsulfonyl)-7H-pyrrolo[2,3-h]quinazoline (IV)

A mixture of 7-(phenylsulfonyl)-1H-pyrrolo[2,3-h]quinazoline-2,4(3H,7H)-dione (I, 1.4 g, 4.1 mmol) and POCl₃ (5 mL) and N,N-dimethylaniline (0.5 mL) was heated at 120° C. for 2 h in microwave oven. The mixture was cooled down to room temperature, and then poured onto ice water with stirring. The resulting solid was collected by filtration to give the title compound 2,4-dichloro-7-(phenylsulfonyl)-7H-pyrrolo[2,3-h]quinazoline IV as off-white solid (1.39 g, 90% yield), MS (ESI) m/z 378.1. Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A method of preparing 1H-pyrrolo[2,3-h]quinazoline-2,4-dione (I): 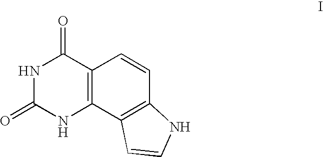
from 4-nitro-1H-indole comprising the steps of reacting 4-nitro-1H-indole with an amine-protecting agent, reducing the nitro group of the product of said reaction to an amine, and reacting said amine with an C₁-C₆alkyl, C₂-C₆alkenyl, C₂-C₆alkynyl, C₆-C₁₄aryl, C₃-C₈cycloalkyl, or C₁-C₉heteroaryl isothiocyanatoformate to yield a 4-thioureido-1H-indole in an aromatic hydrocarbon solvent.

2) The method of claim 1 wherein said aromatic hydrocarbon solvent is toluene.

3) The method of claim 1 wherein the steps of reacting 4-nitro-1H-indole with an amine-protecting agent, reducing the nitro group of product of that reaction to an amine, and reacting said amine with an C₁-C₆alkyl, C₂-C₆alkenyl, C₂-C₆alkynyl, C₆-C₁₄aryl, C₃-C₈cycloalkyl, or C₁-C₉heteroaryl isothiocyanatoformate to yield a 4-thioureido-1H-indole are done without isolation of intermediates.

4) The method of claim 1 wherein said amine-protecting agent transfers a t-butoxycarbonyl group to the indole nitrogen atom.

5) The method of claim 4 wherein said amine-protecting agent is BOC₂O.

6) The method of claim 1 wherein said reducing is done catalytically.

7) The method of claim 6 wherein said catalytic reducing is done with hydrogen.

8) The method of claim 7 wherein said catalytic reducing is done with hydrogen and Pd—C.

9) The method of claim 8 wherein said Pd—C is 5% palladium by weight and is 50% water wet.

10) The method of claim 1 wherein said isothiocyanatoformate is an C₁-C₆alkyl isothiocyanatoformate.

11) The method of claim 10 wherein said C₁-C₆alkyl isothiocyanatoformate is ethyl isothiocyanatoformate.

12) The method of claim 1 further comprising reacting the 4-thioureido-1H-indole with an alkylating agent R²—X, wherein R² is C₁-C₆alkyl and X is a leaving group to give a carbonylamino(C₁-C₆alkylthio)methyleneamino)-1H-indole.

13) The method of claim 12 wherein said R²—X is ethyl iodide.

14) The method of claim 1 further comprising induction of cyclization of the carbonylamino(C₁-C₆alkylthio)methyleneamino)-1H-indole group to give a 2-thio-3H-pyrrolo[2,3-h]quinazolin-4(7H)-one.

15) The method of claim 14 wherein said induction of cyclization of the carbonylamino(C₁-C₆alkylthio)methyleneamino)-1H-indole intermediate group is done thermally.

16) The method of claim 15 wherein the thermal cyclization is done in a eutectic mixture of biphenyl (C₁₂H₁₀) and diphenyl oxide (C₁₂H₁₀O).

17) The method of claim 1 further comprising hydrolysis of the 2-thio-3H-pyrrolo[2,3-h]quinazolin-4(7H)-one to give the final product.

18) The method of claim 17 wherein said hydrolysis is done in an acidic medium.

19) A method of preparing 2,4-dichloro-7H-pyrrolo[2,3-h]quinazoline (II): 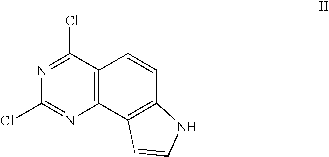
from 1H-pyrrolo[2,3-h]quinazoline-2,4-dione comprising the step of reacting 1H-pyrrolo[2,3-h]quinazoline-2,4-dione with a chlorinating agent, wherein said 1H-pyrrolo[2,3-h]quinazoline-2,4-dione has a purity >97 area % by HPLC.

20) A method of preparing 2,4-dichloro-7-(phenylsulfonyl)-7H-pyrrolo[2,3-h]quinazoline (IV): 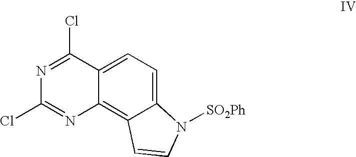
from 7-(phenylsulfonyl)-1H-pyrrolo[2,3-h]quinazoline-2,4(3H,7H)-dione (III): 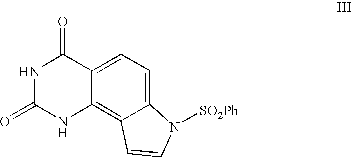
comprising the step of reacting III with a chlorinating agent.

21) The method of claim 20 further comprising the step of hydrolysis of a 2-(C₁-C₆alkylthio)-7-(phenylsulfonyl)-3H-pyrrolo[2,3-h]quinazolin-4(7H)-one to give III.

22) The method of claim 21 further comprising the steps of reacting 4-nitro-1H-indole with benzenesulfonyl chloride, reducing the nitro group of the product of said reaction to an amine, reacting said amine with an C₁-C₆alkyl, C₂-C₆alkenyl, C₂-C₆alkynyl, C₆-C₁₄aryl, C₃-C₈cycloalkyl, or C₁-C₉heteroaryl isothiocyanatoformate to yield a 4-thioureido-1H-indole, reacting the 4-thioureido-1H-indole with an alkylating agent R²—X, wherein R² is C₁-C₆alkyl and X is a leaving group to give a carbonylamino(C₁-C₆alkylthio)methyleneamino)-1H-indole, and induction of cyclization of the carbonylamino(C₁-C₆alkylthio)methyleneamino)-1H-indole to give a 2-(C₁-C₆alkylthio)-7-(phenylsulfonyl)-3H-pyrrolo[2,3-h]quinazolin-4(7H)-one.
